(19) United States
(12) Patent Application Publication
(10) Pub. No.: US 2015/0160163 A1
(43) Pub. Date: Jun. 11, 2015

(54) Hydrogen Concentration Meter

(71) Applicant: Kazuhiro Yamamoto, Tokyo (JP)

(72) Inventors: Kazuhiro Yamamoto, Tokyo (JP); Kazuaki Kato, Ibaraki (JP)

(73) Assignee: Kazuhiro Yamamoto, Tokyo (JP)

(21) Appl. No.: 14/619,803

(22) Filed: Feb. 11, 2015

Related U.S. Application Data
(63) Continuation of application No. PCT/JP2013/004805, filed on Aug. 8, 2013.

(30) Foreign Application Priority Data
Aug. 11, 2012 (JP) 2012-179127

(51) Int. Cl. G01N 27/66; G01N 33/00

(52) U.S. Cl. CPC G01N 27/66; G01N 33/005

(57) ABSTRACT

A hydrogen concentration meter for measuring density of hydrogen in gas, is disclosed having a first electrode and a second electrode. The first electrode is formed of a first metal. The second electrode is formed of a second metal having a work function different from a work function of the first metal. The second electrode faces the first electrode. At least one of the first electrode or the second electrode detects an electrically charged particle generated electrically between the first electrode and the second electrode by a recoil proton generated by an irradiated neutron.

### CROSS-REFERENCE TO RELATED APPLICATION

This application is a continuation of PCT International Application No. PCT/JP2013/004805, filed Aug. 8, 2013, which claims priority under 35 U.S.C. §119 Japanese Patent Application No. JP2012-179127, filed Aug. 11, 2012.

### FIELD OF THE INVENTION

The present invention generally relates to a hydrogen concentration meter, and, more specifically, to a hydrogen concentration meter for use in harsh environments.

### BACKGROUND

Hydrogen concentration meters are often required in harsh environments, such as an inside of a nuclear reactor. Just meters often include a unit for measuring the density of hydrogen and an electric source driving the unit, and follow a method for measuring the density of hydrogen in monitored gas inside of a nuclear reactor, such as described, for example, in Japanese Patent Application No. H6-130177.

For hydrogen concentration meters of this kind, there are situations where they cannot measure the density of hydrogen, because the electric source driving the unit fails in the harsh operating environment. Additionally, some of these conventional hydrogen concentration meters are provided internally with plastic components. The heat resistant temperature of plastic materials is approximately from 60 degrees centigrade to 100 degrees centigrade for general purpose plastic materials used widely (as an example, PMMA; Polymethyl Methacrylate). Therefore, there are situations where hydrogen concentration meters provided internally with components made of general purpose plastic cannot measure the density of hydrogen when the ambient temperature of the hydrogen concentration meters becomes high (500 degrees centigrade, for instance) and the plastic materials become softened and melted.

There is a need for a hydrogen concentration meter which can measure the density of hydrogen in the monitored gas in an environment with high radiation doses, is not provided with any driving electric source, and can measure the density of hydrogen even when the ambient temperature of the hydrogen concentration meter becomes high.

### SUMMARY

A hydrogen concentration meter for measuring density of hydrogen in gas, is disclosed having a first electrode and a second electrode. The first electrode is formed of a first metal. The second electrode is formed of a second metal having a work function different from a work function of the first metal. The second electrode faces the first electrode. At least one of the first electrode or the second electrode detects an electrically charged particle generated electrically between the first electrode and the second electrode by a recoil proton generated by an irradiated neutron.

### BRIEF DESCRIPTION OF THE DRAWINGS

The invention will now be described by way of example, with reference to the accompanying Figures, of which:

FIG. 1 schematically illustrates a structure of a hydrogen concentration meter according to the first embodiment of the present invention;

FIG. 2 schematically illustrates a structure of a hydrogen concentration meter according to the second embodiment of the present invention;

FIG. 3 schematically illustrates a structure of a hydrogen concentration meter according to the third embodiment of the present invention;

FIG. 4 schematically illustrates a structure of a hydrogen concentration meter according to the fourth embodiment of the present invention;

FIG. 5 schematically illustrates a structure of a hydrogen concentration meter according to the fifth embodiment of the present invention.

### DETAILED DESCRIPTION OF THE PREFERRED EMBODIMENT(S)

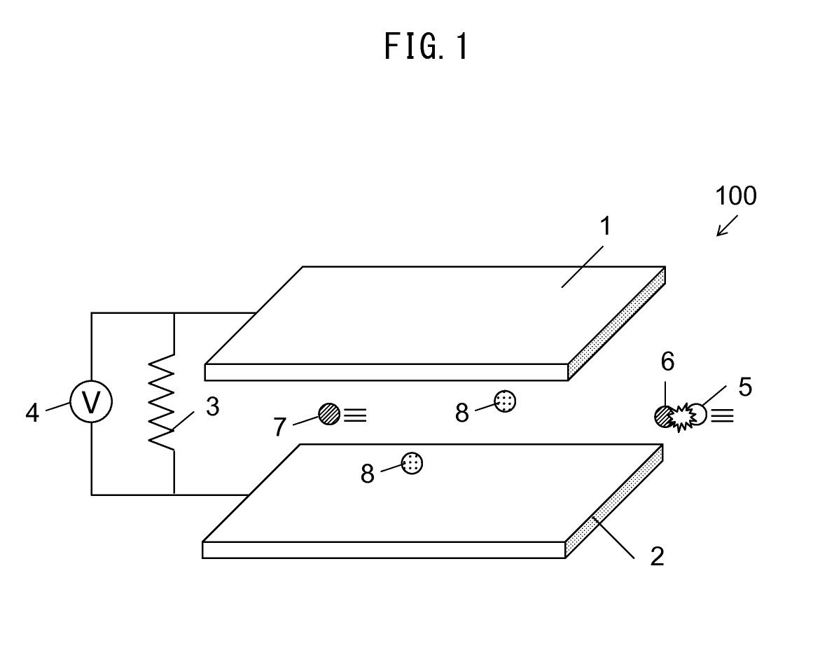

FIG. 1 schematically illustrates a structure of a hydrogen concentration meter according to the first embodiment of the present invention. Hereinafter, while referring to FIG. 1, the structure, operation and effect of a hydrogen concentration meter related to the present embodiment is explained.

A hydrogen concentration meter 100 according to the first embodiment is provided with a first electrode 1 and a second electrode 2 arranged to face the first electrode 1. The first electrode 1 and the second electrode 2 are electrically connected through a resistor 3. The hydrogen concentration meter 100 may further include a voltmeter 4 connected in parallel with the resister 3.

In an embodiment, the first electrode 1 and the second electrode 2 are planar electrodes having an empty space positioned between the first electrode 1 and the second electrode 2, and are positioned to face each other. The space between the first electrode 1 and the second electrode 2 is an open space, where no object is installed which disturbs the flow of air (monitored gas) around the hydrogen concentration meter 100. As such, the air can flow freely between the first electrode 1 and the second electrode 2.

The first electrode 1 is formed of a first metal, and the second electrode 2 is formed of a second metal whose work function is different from that of the first metal. Here, although the first metal and the second metal are selected so that the contact potential difference between their work functions may be large, any pair of metals may be used as long as their work functions differ.

The first metal forming the first electrode 1 may, for example, be one of: Li, Be, Na, Mg, Al, K, Ca, Sc, Ti, V, Cr, Mn, Fe, Co, Ni, Cu, Zn, Ga, Ge, Rb, Sr, Y, Zr, Mo, Tc, Ru, Rh, Pd, Ag, Cd, IN Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, Fr, Ra, Ac, Th, Pa, U, Np, Pu, Am, Cm, Bk, Cf, Es, Fm, Md, No, Lr, Rf, Db, Sg, Bh, Hs, Mt, Ds; or an alloy formed of any combination of the above metals.

The second metal forming the second electrode 1 may, for example, be one of: Li, Be, Na, Mg, Al, K, Ca, Sc, Ti, V, Cr, Mn, Fe, Co, Ni, Cu, Zn, Ga, Ge, Rb, Sr, Y, Zr, Nb, Mo, Tc, Rif, Rh, Pd, Ag, Cd, IN, Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, Fr, Ra, Ac, Th, Pa, U, Np, Pu, Am, Cm, Bk, Cf, Es, Fm, Md, No, Lr, Rf, Db, Sg, Bh, Hs, Mt, Ds; or an alloy formed of any combination of the above metals.

In an embodiment, the resistor 3 and the voltmeter 4 are selected from conventional resistors and conventional voltmeters. Therefore, detailed explanations of the resistor 3 and voltmeter 4 have been omitted.

Hereinafter, a method of measuring the density of hydrogen by using the hydrogen concentration meter 100 related to the first embodiment is explained.

The hydrogen concentration meter 100 determines the density of hydrogen in monitored gas by utilizing ionization power of a recoil proton 7 generated by elastic scattering of a neutron 5 and a proton (hydrogen atomic nuclei) 6. Therefore, it is assumed that the hydrogen concentration meter 100 is used in an environment where a certain amount of neutron 5 exist around the hydrogen concentration meter 100, such as inside of a nuclear reactor, in locations having high radiation levels, and in locations where a neutron source is available.

Firstly, it is supposed that a certain amount of hydrogen exists in the monitored gas existing between the first electrode 1 and the second electrode 2 arranged to face each other. A proton 6 that forms hydrogen is caused to become a recoil proton 7 by a neutron 5 emitted from a neutron source (not shown), such as radioactive isotope including Cf-252 and Am-242+Be. If the amount of neutron 5 irradiated to proton 6 is assumed to be fixed (or, normalized), the amount of recoil proton 7 is directly proportional to the amount of hydrogen existing between the first electrode 1 and the second electrode 2, namely, the density of hydrogen in the monitored gas.

When the recoil proton 7 is generated, the proton 6 receives kinetic energy from the neutron 5 with high efficiency. This is because the mass of the proton 6 is smaller than that of other atomic nucleus, such as that of oxygen and nitrogen. The recoil proton 7, having received kinetic energy from the neutron 5, can easily ionize the monitored gas (regardless of kinds of molecules and atoms forming the monitored gas) existing between the first electrode 1 and the second electrode 2, and generates a charged particle 8. Because the amount of generated recoil proton 7 is directly proportional to the density of hydrogen in the monitored gas, as described above, the amount of the generated charged particle 8 is directly proportional to the density of hydrogen in the monitored gas.

The hydrogen concentration meter 100 detects generated charged particles 8 by utilizing contact potential difference occurring between the first electrode 1 and the second electrode 2. As described above, the first electrode 1 and the second electrode 2 are individually formed of metals with different work functions. If metals of two kinds having work functions different from each other are electrically connected through a metal wire, contact potential difference occurs between the first electrode 1 and the second electrode 2. By utilizing the potential difference, the charged particle 8 generated by recoil proton 7 can be detected with either the first electrode 1 or the second electrode 2. Additionally, the amount of generated charged particle 8, namely, an amount of electric charge, is measured as electromotive force generated between the first electrode 1 and the second electrode 2 with the voltmeter 4.

By comparing the voltage actually measured, corresponding to the quantity correlated with ionization power of recoil proton 7, with voltage values measured and recorded in a conversion table in advance, the density of hydrogen in the monitored gas can be determined. The density of hydrogen in the monitored gas is the density of hydrogen between the first electrode 1 and the second electrode 2.

One of ordinary skill in the art would appreciate that the embodiment of the hydrogen concentration meter 100, where the first electrode 1 and the second electrode 2 are planar electrodes, is merely exemplary, and is not limiting. The shapes of the first electrode 1 and the second electrode 2 can be adjusted according to the shape of the location where the hydrogen concentration meter 100 is installed. For example, when the hydrogen concentration meter 100 is installed in a ceiling of a building, the shapes of the electrode 1 and the electrode 2 can be changed according to the shape of the ceiling of the building. In a further example, when the hydrogen concentration meter 100 is installed inside of a nuclear reactor, the shapes of the electrode 1 and the electrode 2 can be changed according to the shape of the nuclear reactor.

Further, one of ordinary skill in the art would appreciate that the first embodiment of the hydrogen concentration meter 100, where the first electrode 1 and the second electrode 2 are formed of metals or alloys, is merely exemplary, and is not limiting. The first electrode 1 and the second electrode 2 may be formed of inorganic material having electroconductivity or organic material having electroconductivity, instead of metals or alloys as mentioned above. For example, inorganic material having electroconductivity includes concrete having electroconductivity, and organic material having electroconductivity includes resin material having electroconductivity.

As described above, the hydrogen concentration meter 100 measures the density of hydrogen by deriving the density of hydrogen from the amount of charged particle 8 generated by recoil proton 7, by detection with the first electrode 1 or the second electrode 2. Thus, the hydrogen concentration meter 100 measures the density of hydrogen by utilizing ionization power of recoil proton 7. Therefore, the hydrogen concentration meter 100 can measure the density of hydrogen in monitored gas in an environment with high radiation dose.

In an embodiment, the hydrogen concentration meter 100 measures the amount of charged particle 8 through the amount of electric charge generated by recoil proton 7 by utilizing contact potential difference occurring between the first electrode 1 and the second electrode 2. Therefore, the hydrogen concentration meter 100 can be configured to measure the density of hydrogen in the monitored gas without utilizing any driving electric source.

Since the primary components of the hydrogen concentration meter 100 are pairs of electrodes 1 and 2 made of metal, the hydrogen concentration meter 100 may be easily be made without components made of materials having low softening/melting points. As such, the hydrogen concentration meter 100 is able to measure the density of hydrogen, even when the ambient temperature of the hydrogen concentration meter 100 becomes high. In an embodiment, such elevated temperatures are greater than or equal to 500 degrees centigrade. In an embodiment, such elevated temperatures are greater than or equal to 1000 degrees centigrade, depending upon the metal selected as the electrode material.

Accordingly, the hydrogen concentration meter 100 can measure the density of hydrogen in monitored gas, even in harsh environments and in places inhospitable to humans. In an embodiment, when the hydrogen concentration meter 100 is installed in a building for a nuclear reactor, the hydrogen concentration meter 100 is installed inside the building and near the ceiling of the building.

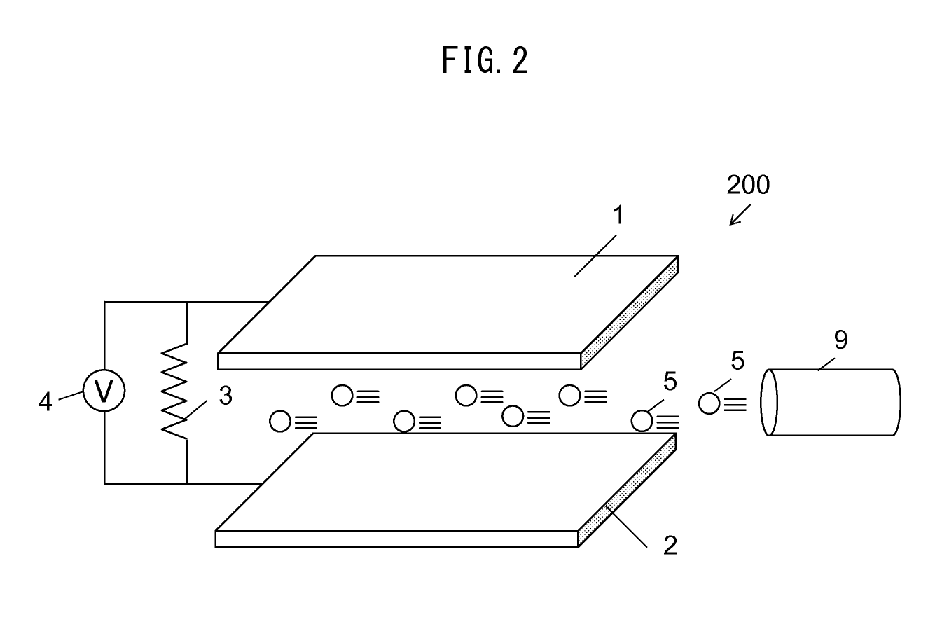

FIG. 2 schematically illustrates a structure of a hydrogen concentration meter 200 according to the second embodiment. In the second embodiment, the hydrogen concentration meter 200 has substantially the same structure as that of the hydrogen concentration meter 100 related to the first embodiment. However, the hydrogen concentration meter 200 differs in its structure from the hydrogen concentration meter 100 in that it is provided with a neutron source 200, so that neutrons 5 may pass between the first electrode 1 and the second electrode 2. Therefore, only the neutron source 200 is explained, and the common remaining parts are omitted from discussion. In FIG. 2, the same numbers are assigned to the same components as components explained in FIG. 1. Additionally, although FIG. 2 omits illustrations of the recoil proton 7 and charged particle 8, it is assumed, similarly to the first embodiment, that the recoil proton 7 is generated by a neutron 5, and the charged particle 8 is generated by the recoil proton 7.

The neutron source 9 is positioned such that the neutron 5 may pass between the first electrode 1 and the second electrode 2 in an extending direction of the first electrode 1 and the second electrode 2. The position of the neutron source 9 is irrelevant as long as the neutron 5 may pass between the first electrode 1 and the second electrode 2 in extending direction of the first electrode 1 and the second electrode 2. Embodiments of the neutron source 9 include radioactive isotopes, such as Cf-252 and Am-242+Be, whose energy is approximately several MeV, as well as a proton accelerator, such as a linear accelerator, generating neutrons having an energy of approximately 14 MeV by utilizing nuclear reaction D (p, n). When the radioactive isotope is utilized as the neutron source 9, the radioactive isotope may be embedded in parts of first and second electrodes 1,2, or may be coated on parts of the surface of the electrodes. In addition, the radioactive isotope may be coated on the entire surface of the first and second electrodes 1,2 if electric conductivity of the electrodes is not decreased.

The hydrogen concentration meter 200 can increase the amount of neutron 5 passing between the first electrode 1 and the second electrode 2 in extending direction of the first electrode 1 and the second electrode 2 compared to a hydrogen concentration meter without neutron source. Therefore, the amount of recoil proton 7 between the first electrode 1 and the second electrode 2 can be increased, and the amount of charged particle 8 can be increased. Consequently, the hydrogen concentration meter 200 can measure the density of hydrogen in monitored gas in higher sensitivity.

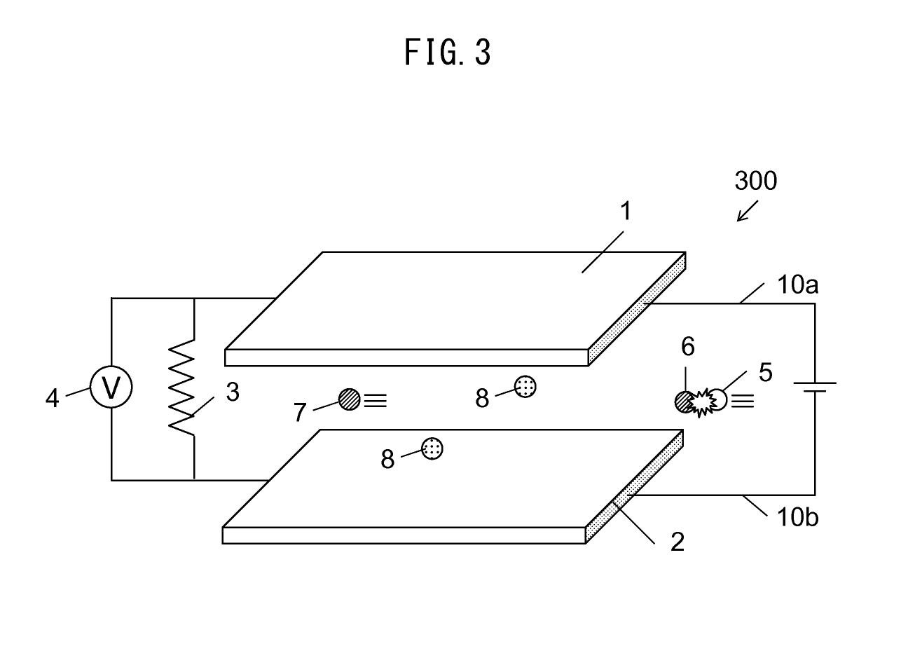

FIG. 3 schematically illustrates a structure of a hydrogen concentration meter 300 according to the third embodiment of the present invention. In the third embodiment, the hydrogen concentration meter 300 has substantially the same structure as that of the hydrogen concentration meter 100 related to the first embodiment. However, the hydrogen concentration meter 300 differs in its structure from the hydrogen concentration meter 100 in that voltage is separately applied to the first electrode 1 and the second electrode 2. Therefore, only the voltage is explained, and the common remaining parts are omitted from further discussion. In FIG. 3, the same numbers are assigned to the same components as components explained in FIG. 1.

A first metal wiring 10a is connected to the first electrode 1, so that voltage may be applied to the first electrode 1 through the first metal wiring 10a. In addition, as with the first electrode 1, a second metal wiring 10b is connected to the second electrode 2, so that voltage may be applied to the second electrode 2 through the second metal wiring 10b.

The first metal wiring 10a connected to the first electrode 1 may be made of the same metal as the second metal wiring 10b connected to the second electrode 2, or may be made of a different metal from the second metal wiring 10b.

Although FIG. 3 illustrates that positive potential is applied to the first electrode 1 and negative potential is applied to the second electrode 2, polarities of potentials for electrodes may be exchanged. This means that negative potential can be applied to the first electrode 1 and positive potential can be applied to the second electrode 2. It should be noted that the above mentioned voltage applied to the electrodes 1 and 2 may be chosen arbitrarily if stability of output with respect to variation of applied voltage is considered unimportant, but, otherwise, the voltage should be preferably set in a “saturation range” in V-I characteristics, and the range can be obtained easily experimentally. In an embodiment, the voltage is set in a range between several volts to several tens of volts, but, in other embodiments, the voltage may be more or less than the range.

Therefore, the hydrogen concentration meter 300 of the third embodiment, with voltage applied to the first electrode 1 and the second electrode 2, can capture charged particles 8, ionized by the recoil proton 7, with the first electrode 1 or the second electrode 2 more efficiently than a hydrogen concentration meter without the voltage applied to the first electrode 1 and the second electrode 2. Consequently, the hydrogen concentration meter 300 can measure the density of hydrogen with higher sensitivity.

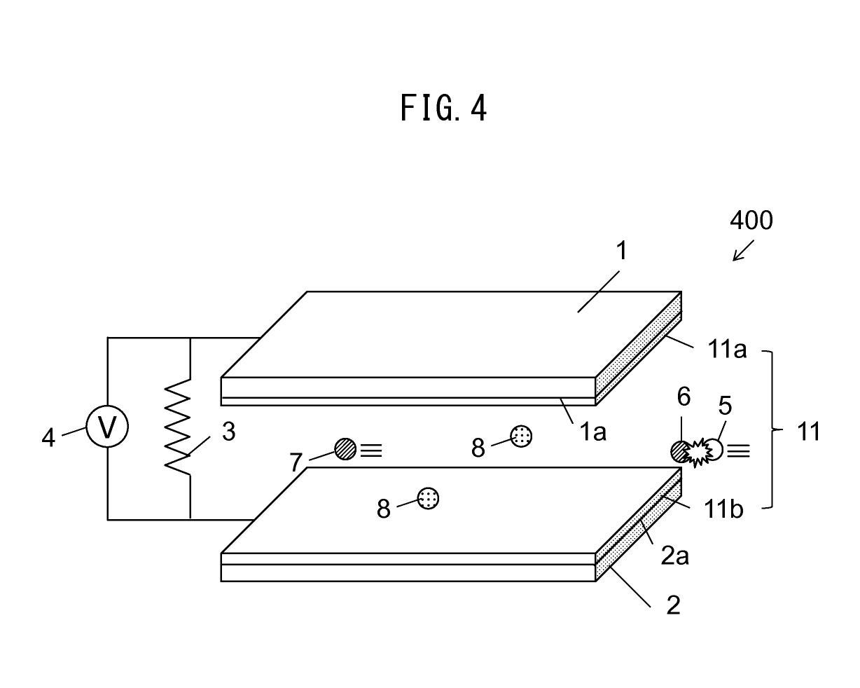

FIG. 4 schematically illustrates a structure of a hydrogen concentration meter 400 according to the fourth embodiment of the present invention. In the fourth embodiment, the hydrogen concentration meter 400 has substantially the same structure as that of the hydrogen concentration meter 100 related to the first embodiment. However, the hydrogen concentration meter 400 differs in its structure from the hydrogen concentration meter 100 in that surfaces of the first electrode 1 and the second electrode 2 are covered with metal thin films 11. Therefore, only the metal thin films 11 are explained, and the common remaining parts are omitted from further rdiscussion. In FIG. 4, the same numbers are assigned to the same components as components explained in FIG. 1.

In the fourth embodiment of FIG. 4, the first electrode 1 has a first surface 1a facing the second electrode 2, and being covered with a first metal thin film 11a. The second electrode 2 has a second surface 2a facing the first electrode 1, and being covered with a second metal thin film 11b. In an embodiment, the metal used for the first metal thin film 11a and the metal used for the second metal thin film 11b are selected so that the contact potential difference between their work functions is large, although any pair of metals may be used as long as their work functions differ.

The first metal thin film may, for example, be made of one of: Li, Be, Na, Mg, Al, K, Ca, Sc, Ti, V, Cr, Mn, Fe, Co, Ni, Cu, Zn, Ga, Ge, Rb, Sr, Y, Zr, Nb, Mo, Tc, Ru, Rh, Pd, Ag, Cd, IN, Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, Fr, Ra, Ac, Th, Pa, U,Np, Pu, Am, Cm, Bk, Cf, Es, Fm, Md, No, Lr, Rf, Db, Sg, Bh, Hs, Mt, Ds; or an alloy formed of any combination of the above metals.

In addition, the second metal thin film may, for example, be made of one of: Li, Be, Na, Mg, Al, K, Ca, Sc, Ti, V, Cr, Mn, Fe, Co, Ni, Cu, Zn, Ga, Ge, Rb, Sr, Y, Zr, Nb, Mo, Tc, Ru, Rh, Pd, Ag, Cd, IN, Sn, Sb, Cs, Ba, La, Ce, Pr, Nd, Pm, Sm, Eu, Gd, Tb, Dy, Ho, Er, Tm, Yb, Lu, Hf, Ta, W, Re, Os, Ir, Pt, Au, Hg, Tl, Pb, Bi, Po, Fr, Ra, Ac, Th, Pa, U, Np, Pu, Am, Cm, Bk, Cf, Es, Fm, Md, No, Lr, Rf, Db, Sg, Bh, Hs, Mt, Ds; or an alloy formed of any combination of the above metals.

The fourth embodiment can also cause contact potential difference to occur between the first metal thin film 11a of the first electrode 1 and the second metal thin film 11b of the second electrode 2. Therefore, the fourth embodiment has substantially the same effects as the first embodiment.

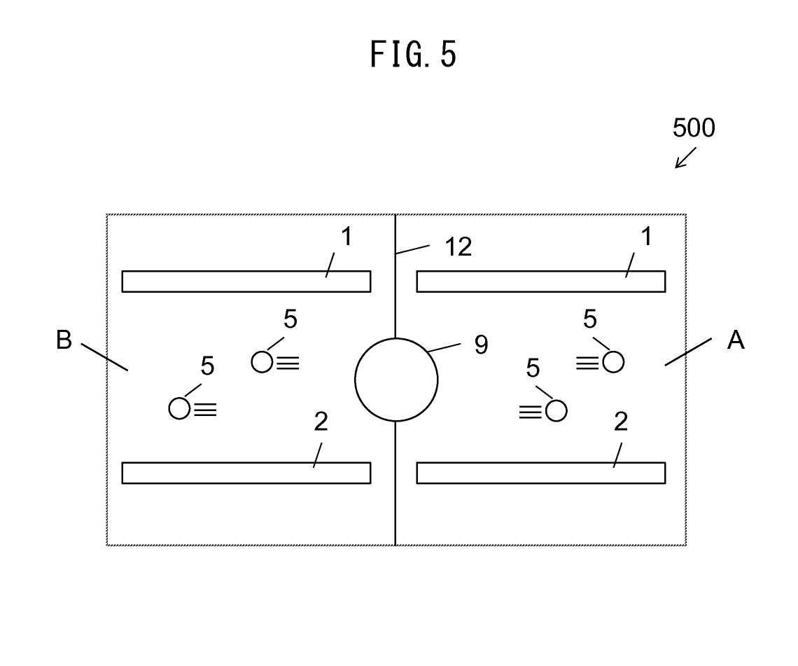

FIG. 5 schematically illustrates a structure of a hydrogen concentration meter 500 according to the fifth embodiment of the present invention. In the fifth embodiment, the hydrogen concentration meter 500 has two adjacent containers A and B. Hydrogen concentration meters 100 are respectively positioned in the containers A and B. Therefore, for the fifth embodiment, only the containers A and B are explained, and the common remaining parts are omitted from further discussion. In FIG. 5, the same numbers are assigned to the same components as components explained in FIG. 1.

The hydrogen concentration meter 500 has two adjacent containers A and B, as FIG. 5 illustrates. An inner space of the container A, shown in a right side of FIG. 5, is a closed space. The closed space is filled completely with only nitrogen and oxygen, and does not contain hydrogen. In contrast, an inner space of the container B, shown in a left side of FIG. 5, is an open space. As such, the container B has openings (not shown) on both front and back sides, and the monitored gas, such as one containing hydrogen, may pass through the container B from the front side to the back side.

Pairs of the first electrode 1 and second electrode 2 are respectively positioned in the containers A and B, as discussed above for the hydrogen concentration meter 100. In addition, a neutron source 9 is positioned in a partition wall 12 which separates the containers A and B. The neutron source 9 emits the same amount of neutrons into the containers A and B. Since the mechanism whereby the emitted neutron generates recoil protons 7 and the recoil protons 7 generate charged particles 8 is explained in the first embodiment, its explanation is omitted in the following description. In addition, FIG. 5 omits illustrations of the recoil proton 7 and charged particle 8.

The hydrogen concentration meter 500 can utilize the electromotive force generated between the pair of electrodes 1 and 2 positioned in the container A as a reference of electromotive force (hereinafter referred to as “reference electromotive force”). By comparing the reference electromotive force and an electromotive force generated between a pair of electrodes 1 and 2 positioned in the container B, the density of hydrogen in monitored gas can be measured more accurately.

It should be noted that the present embodiment is explained with an arrangement where the same amount of neutron is emitted into the containers A and B, but the present embodiment is not limited thereto. For example, even when neutron 5 emitted toward the container A may be obstructed and neutron 5 is emitted only into the container B, the same effect as those with the above embodiment is achieved. It should be noted that when emitted neutron 5 is obstructed, members preventing permeation of neutron is used, for example.

A hydrogen concentration meter related to a variation of the above mentioned embodiments is a hydrogen concentration meter (not illustrated in the figures) which combines a hydrogen concentration meter 100 related to the first embodiment and a hydrogen concentration meter 300 related to the third embodiment. The hydrogen concentration meter related to the present variation may be positioned near the ceiling of a building for a nuclear reactor, as an example. The hydrogen concentration meter related to the present variation can measure the density of hydrogen in monitored gas with the hydrogen concentration meter 300, which has a relatively higher sensitivity, when radiation dose is low. In addition, the present hydrogen concentration meter can measure the density of hydrogen in monitored gas with the hydrogen concentration meter 100 even when the temperature and/or radiation dose in the environment around the present hydrogen concentration meter becomes high, and thus the hydrogen concentration meter 300 can not be utilized.

As described above, the hydrogen concentration meter related to the present variation can measure the density of hydrogen in both an ordinary measurement environment and a harsh measurement environment.

The present invention may be embodied in other specific forms without departing from the spirit or essential characteristics thereof The embodiments disclosed in this application are to be considered in all respects as illustrative and not restrictive, the scope of the invention being indicated by the appended claims rather than by the foregoing description, all changes that come within the meaning and range of equivalency of the claims are intended to be embraced therein.

What is claimed is:

1. A hydrogen concentration meter for measuring density of hydrogen in gas, comprising:
a first electrode formed of a first metal;
a second electrode formed of a second metal having a work function different from a work function of the first metal, the second electrode facing the first electrode; wherein, at least one of the first electrode or the second electrode detects an electrically charged particle generated electrically between the first electrode and the second electrode by a recoil proton generated by an irradiated neutron.

2. A hydrogen concentration meter according to claim 1, further comprising a neutron source positioned between the first electrode and the second electrode, wherein, a neutron passes between the first electrode and the second electrode in an extending direction of the first electrode and the second electrode.

3. A hydrogen concentration meter according to claim 2, wherein, voltage is applied to the first electrode and the second electrode.
